(19) United States
(12) Patent Application Publication
(10) Pub. No.: US 2026/0309674 A1
(43) Pub. Date: Oct. 8, 2026

(54) FLUID CELL DEVICE AND METHOD FOR MEASURING DRILLING MUD PROPERTIES

(71) Applicant: Chevron U.S.A. Inc., San Ramon, CA (US)

(72) Inventors: Alexei Olegovich Bolshakov, Pearland, TX (US); Kristoffer Thomas Walker, Kingwood, TX (US)

(21) Appl. No.: 19/446,705

(22) Filed: Jan. 12, 2026

Related U.S. Application Data
(60) Provisional application No. 63/744,562, filed on Jan. 13, 2025.

(51) Int. Cl. G01N 29/024; E21B 44/00; E21B 49/00; G01N 33/28

(52) U.S. Cl. CPC G01N 29/024; E21B 44/00; E21B 49/005; G01N 33/2823; G01N 2291/011; G01N 2291/018; G01N 2291/0226

(57) ABSTRACT

A fluid cell device and method are described for measuring properties of drilling mud in a borehole in an earth formation. The fluid cell device includes a housing, a first chamber, a second chamber adjacent to the first chamber, and a plate located between the first chamber and the second chamber. The drilling mud from the borehole flows into the first chamber and the second chamber. A transducer transmits an acoustic signal across the first chamber toward the plate. Reflections and reverberations responsive to the acoustic signal are analyzed to determine properties of the drilling mud including mud velocity and mud impedance.

### CROSS-REFERENCE TO RELATED APPLICATIONS

The present application claims priority to U.S. Provisional Patent Application No. 63/744,562 filed Jan. 13, 2025, the entire content of which is incorporated herein by reference.

### TECHNICAL FIELD

The disclosed embodiments relate generally to a device and techniques for measuring properties of drilling mud used for hydrocarbon production from subsurface reservoirs. In particular, the device and methods enable simultaneous measurement of drilling mud acoustic velocity, impedance, and attenuation.

### BACKGROUND

Drilling mud, a crucial component in the oil and gas industry, plays a significant role in various operations such as drilling, completion, production, and shut in. Its functions are manifold and include, but are not limited to: a) applying hydrostatic pressure to prevent the intrusion of formation fluids into the wellbore; b) removing drill cuttings during drilling and suspending them when drilling is halted; c) cooling the drill bit and clearing cuttings from beneath it during drilling; d) regulating well pressure and stabilizing the formation; and e) cooling and lubricating the wellbore.

Moreover, drilling mud serves as a conduit from the formation to the tools in the well and to the surface. Understanding the properties of the drilling mud in the well is vital for several reasons: a) identifying the points of formation fluid entry into the well and the properties of the formation fluid; b) tracking the loss of drilling mud additives into the formation and pinpointing where these losses occurred; c) assisting both Logging While Drilling (LWD) and Wire Line (WL) logging tools with measurements and interpretation, as drilling mud properties are crucial for interpreting resistivity, dielectric, and acoustic tool measurements.

The fluid cells currently in use in the industry are designed to measure only the acoustic velocity of the drilling mud. This design and its operating principles are illustrated in FIG. 1. The fluid cell, with a width denoted as D1, is exposed to the drilling mud in the wellbore, and has a transducer, T1, on one side. This transducer emits a short pulse that travels to the cell's opposite side and back. It is important to note that in all the following discussions we use plane acoustic wave modeling. The velocity of the drilling mud is then calculated using the following formula:

$${V}_{m} = \frac{2 D 1}{TF 1 - Δ T 1} . \qquad ( 1 )$$

In this equation, Vₘ is the mud velocity, D1 is the width of the fluid cell, TF1 is the “time of flight” for the acoustic signal travelling to the cell's opposite side and back, ΔT1 is the time offset that accounts for the noise threshold and/or the time needed for the wavelet to accumulate enough energy to rise above the noise level (which can be obtained from calibration). It is worth noting that the transducer both emits the wavelet and captures the reflected energy. Special care should be taken to suppress the electromagnetic energy surge created by the firing pulse, ensuring that the reflected energy is recorded with minimal interference from the firing pulse.

There are conventional acoustic devices to measure ultrasound velocity in drilling mud. The measurement of mud velocity in these conventional devices is done based on the time of propagation of ultrasonic wave through mud over a fixed distance or time difference in time of flight of acoustic sound propagating along two different distances. Additionally, some conventional methods measure mud attenuation based on amplitude reduction of the first arrivals of acoustic signals that traveled different distances.

Other conventional methods may use “plate” (casing) reverberations for cement evaluation. However, these do not rely on the fact that materials on either side of the plate/casing have the same properties and are forced to assume mud slowness impedance to calculate the impedance of material on the other side of the plate.

In ultrasonic cement evaluation and casing inspection, the tool typically measures mud slowness only and assumes a nominal mud density to calculate mud acoustic properties. This approach simplifies the process but introduces uncertainty because actual mud density can vary with composition, temperature, and pressure. As a result, reflection coefficients and interface modeling may be inaccurate, affecting interpretation of cement bond quality and casing integrity. Knowing the true mud acoustic impedance would significantly improve these measurements. Accurate impedance allows precise calculation of reflection and transmission at the mud-casing and casing-cement interfaces, leading to better calibration of ultrasonic tools and more reliable detection of issues such as micro-annuli, poor bonding, or casing defects. Direct measurement of mud acoustic impedance instead of assuming it improves accuracy and strengthens confidence in casing integrity assessments and cement bond log interpretation.

Accordingly, prior art approaches have relied upon estimates of mud density and mud attenuation. None of the prior art teaches how to determine the mud density or mud attenuation.

There exists a need for devices and methods that can measure additional properties of the drilling mud, including acoustic velocity, impedance, and attenuation.

### SUMMARY

The present disclosure is directed to a fluid cell device and method for using the fluid cell device to measure properties of a drilling mud located in a borehole in an earth formation.

In accordance with certain example embodiments, the fluid cell device may include: a) a housing, b) a first chamber within the housing and having a first width, the first chamber configured to receive a portion of the drilling mud c) a second chamber within the housing and adjacent to the first chamber, the second chamber having a second width and configured to receive a portion of the drilling mud, d) a plate disposed between the first chamber and the second chamber, wherein the plate is located at a distal end of the first chamber and a proximal end of the second chamber, and e) a first transducer disposed at a proximal end of the first chamber, wherein the first transducer directs an acoustic signal toward a proximal surface of the plate.

In accordance with additional example embodiments, a method is provided. The method may include: a) deploying a fluid cell device into drilling mud in a borehole, and b) analyzing recorded reflections and reverberations responsive to the acoustic signal to determine a mud velocity and a mud impedance of the drilling mud. The fluid cell device may include: a) a housing, b) a first chamber within the housing and having a first width, the first chamber configured to receive a portion of the drilling mud c) a second chamber within the housing and adjacent to the first chamber, the second chamber having a second width and configured to receive a portion of the drilling mud, d) a plate disposed between the first chamber and the second chamber, wherein the plate is located at a distal end of the first chamber and a proximal end of the second chamber, and e) a first transducer disposed at a proximal end of the first chamber, wherein the first transducer directs an acoustic signal toward a proximal surface of the plate.

The foregoing embodiments are non-limiting examples and other aspects and embodiments will be described herein. The foregoing summary is provided to introduce various concepts in a simplified form that are further described below in the detailed description. This summary is not intended to identify required or essential features of the claimed subject matter nor is the summary intended to limit the scope of the claimed subject matter.

### BRIEF DESCRIPTION OF THE DRAWINGS

FIG. 1 illustrates a conceptual design and principles of operation of a conventional fluid cell device currently used in the oil and gas industry;

FIG. 2 illustrates an embodiment of a design of a fluid cell device in accordance with the example embodiments provided herein;

FIG. 3 illustrates amplitudes of reflection and reverberations in the fluid cell device shown in FIG. 2 in accordance with the example embodiments provided herein;

FIG. 4 illustrates reflection and reverberations in the presence of the plate in accordance with the example embodiments provided herein;

FIG. 5 illustrates separation of the reflection and reverberations in accordance with the example embodiments provided herein;

FIG. 6 illustrates signal received on a transducer (T2) in accordance with the example embodiments provided herein;

FIG. 7 illustrates choosing the optimal relationship between the plate thickness and the transducer frequency in accordance with the example embodiments provided herein; and

FIG. 8 illustrates using a fluid cell device in a borehole in accordance with the example embodiments provided herein.

Like reference numerals refer to corresponding parts throughout the drawings.

### DETAILED DESCRIPTION OF EMBODIMENTS

Described below are a device and techniques for measuring properties of drilling mud used for hydrocarbon production from subsurface reservoirs. In particular, the device and methods enable simultaneous measurement of drilling mud acoustic velocity, impedance, and attenuation. While traditional fluid cells in the industry can only measure mud acoustic velocity, this invention introduces a fluid cell design that enables simultaneous measurement of drilling mud acoustic velocity, impedance, and attenuation in both Logging While Drilling (LWD) and Wire Line (WL) environments. Furthermore, the proposed design enhances the reliability of these measurements through redundancy.

Reference will now be made in detail to various embodiments, examples of which are illustrated in the accompanying drawings. In the following detailed description, numerous specific details are set forth in order to provide a thorough understanding of the present disclosure and the embodiments described herein. However, embodiments described herein may be practiced without these specific details. In other instances, well-known methods, procedures, components, and mechanical apparatus have not been described in detail so as not to unnecessarily obscure aspects of the embodiments.

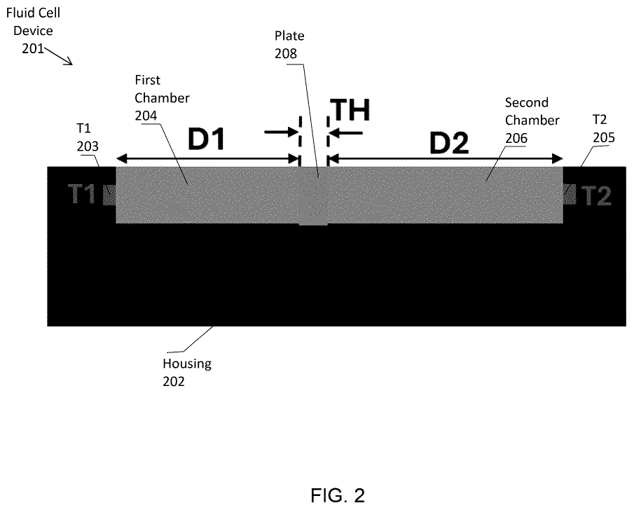

An example of a fluid cell device in accordance with the embodiments herein is shown in FIG. 2. The example fluid cell device 201 comprises a first chamber 204 having first width D1 and second chamber 206 having a second width D2. While second width D2 is shown as being greater than first width D1 in the example of FIG. 2, in alternate embodiments the two widths can be equal. The first chamber 204 and the second chamber 206 are separated by a plate 208 having a thickness TH. In addition to the required transducer T1 203, the example fluid cell device 201 includes an optional second transducer T2 205. In certain embodiments, the optional second transducer T2 205 may function as a receiver only.

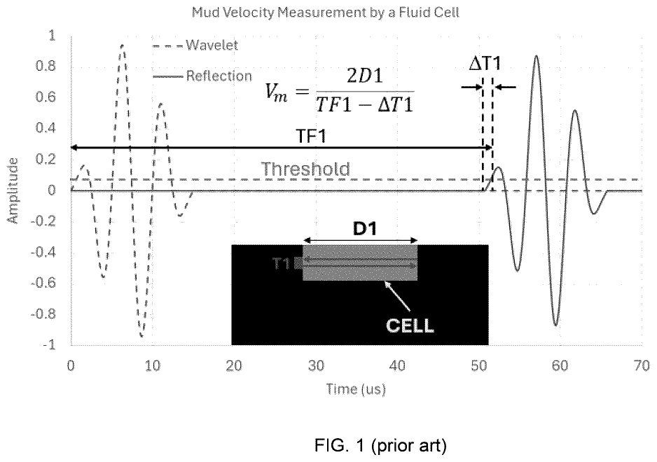

Both the first and second chambers 204, 206 of this fluid cell device 201 are open to the drilling mud present in the wellbore. The principle of operation of the example fluid cell device relies on the presence of the same fluid on either side of the plate. When processing for mud velocity and impedance we use the following two measurements: a) plate reflection, which allows to process for the mud velocity (similar to what is shown in FIG. 1 and equation (1)), and b) plate reverberations (depending on the reflection coefficient between plate and mud), which allows to process for the mud impedance. Additionally, if the two chambers of the fluid cell device have different widths (D1≠D2), difference in reverberations' amplitudes measured by transducers T1 and T2 allows for the mud attenuation estimation. We now define the expressions that will be used in the discussion to follow.

If the acoustic velocities of the plate (p) and mud (m) are Vₚ and Vₘ and the densities of the plate and mud are ρₚ and ρₘ, then their corresponding acoustic impedances have the following form:

$${Z}_{p} = {ρ}_{p} {V}_{p} , {Z}_{m} = {ρ}_{m} {V}_{m} . \qquad ( 2 )$$

The reflection and transmission coefficients for the normally incident plane wave at the plate-mud boundary will have the following form:

$${R}_{mp} = \frac{{Z}_{p} - {Z}_{m}}{{Z}_{p} + {Z}_{m}} , {R}_{pm} = - {R}_{mp} , R = \vert {R}_{mp} \vert , \\ {T}_{mp} = 1 + {R}_{mp} = \frac{2 {Z}_{p}}{{Z}_{p} + {Z}_{m}} , {T}_{pm} = 1 + {R}_{pm} = \frac{2 {Z}_{m}}{{Z}_{p} + {Z}_{m}} , \qquad ( 3 )$$

where R is the absolute value of the reflection coefficient, R and T with subscripts are reflection and transmission coefficients when the acoustic wave travels from mud to plate (subscript “mp”) and from plate to mud (subscript “pm”).

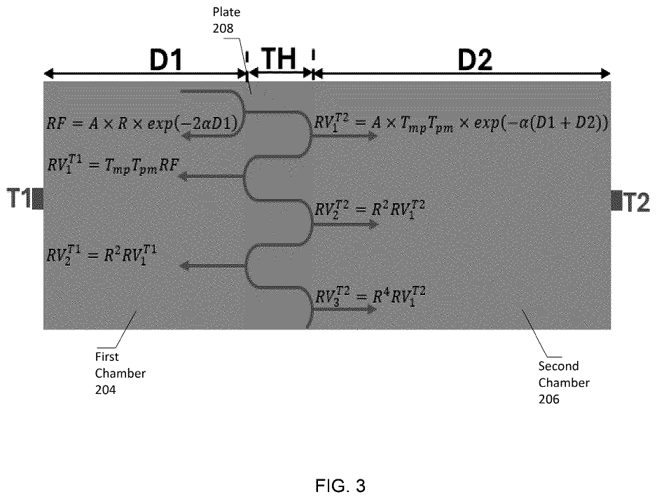

We use expressions defined in (3) to determine the amplitudes of the plate reflection and intraplate reverberations (see FIG. 3). When transducer T1 fires, the amplitude of the reflected energy RF registered on transducer T1 is equal to:

$$RF = A \times R \times exp ( - 2 α D 1 ) , \qquad ( 4 )$$

where A is the amplitude of the wavelet fired by transducer T1 and α is the mud attenuation coefficient. The amplitude of the first reverberation

$${RV}_{1}^{T 1}$$

registered by transducer T1, which can also be thought of as the reflection from the right interface of the plate with a reflection coefficient that has the same magnitude as R, is:

$${RV}_{1}^{T 1} = {T}_{mp} {T}_{pm} RF = A \times R \times {T}_{mp} {T}_{pm} \times exp ( - 2 α D 1 ) . \qquad ( 5 )$$

The amplitude of the first wave (reverberation)

$${RV}_{1}^{T 2}$$

arriving at transducer T2 is equal to:

$${RV}_{1}^{T 2} = A \times {T}_{mp} {T}_{pm} \times exp ( - α ( D 1 + D 2 ) ) . \qquad ( 6 )$$

We also note that the relationship between the reverberations can be expressed in the following form:

$${RV}_{n}^{T 1} = {R}^{2 ( n - 1 )} {RV}_{1}^{T 1} , {RV}_{n}^{T 2} = {R}^{2 ( n - 1 )} {RV}_{1}^{T 2} , n > 1. \qquad ( 7 )$$

From (7) we see that every time the wave travels back and forth through the plate it reflects twice with a coefficient of R, dropping the amplitude by R². Thus, the reverberation amplitude has the following general form:

$$E ( t ) = a \times {R}^{\frac{t}{TH * {V}_{p}}} , t > {t}_{0} , \qquad ( 8 )$$

where a is the reverberations' amplitude and t₀ is the time when the first “unobstructed” reverberations' energy peak arrives. We note that reverberations recorded on transducer T1 (firing transducer) may be obstructed by the reflection RF, especially when the firing wavelet is long while in general a larger portion of the reverberation should be available on transducer T2 (non-firing transducer). We also note that the time difference in reverberation arrivals, T₁₂, between transducers T1 and T2 is:

$${T}_{12} = ( D 1 - D2 ) / {V}_{m} + TH / {V}_{p} , \qquad ( 9 )$$

where if T₁₂>0 then the reverberations arrive first to transducer T2 and if T₁₂<0 then reverberations arrive first to transducer T1.
Fluid Cell Device Design with One Transducer

If only one transducer is present, the mud attenuation measurement is not possible. In this case, the mud velocity can be measured using (1). The mud impedance can be extracted by fitting the reverberations' energy decay with equation (8) which gives reflection coefficient R and then using the following equation:

$${Z}_{m} = {Z}_{p} \frac{1 - R}{1 + R} . \qquad ( 10 )$$

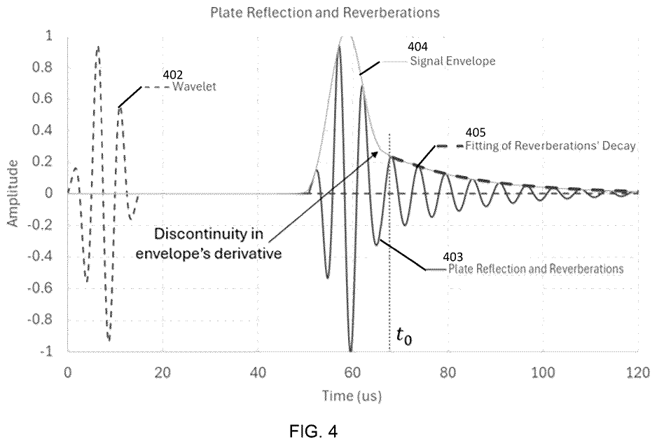

The fitting of the reverberations' energy decay can be done by fitting the envelope of the reverberations as demonstrated in FIG. 4. Alternatively, one may consider taking the local extremums of the waveforms at the top of the crest and bottom of the troughs correspondingly, taking the absolute value of these and fitting the corresponding points with equation (8). Shown in this figure is the incident acoustic wavelet (dashed line 402), signal recorded on transducer T1 consisting of reflection RF and plate reverberations (solid line 403), envelope of that signal (solid line 404) and fitting of reverberations part of the envelope (dashed line 405) using (8). Comparing FIGS. 1 and 4 (which used the same wavelet, material properties and D1 distance) we note that the signal recorded on transmitter T1 in this case has a longer “tail” as it combines both plate reflection and reverberations.

Special care should be taken when choosing the time t₀ used in equation (7). One “natural” choice would be to set it to the time instance where the signal envelope's derivative is discontinuous. However, our investigations demonstrated that it is incorrect as what we are looking for is the decaying part of the recorded signal's envelope that corresponds to reverberations only.

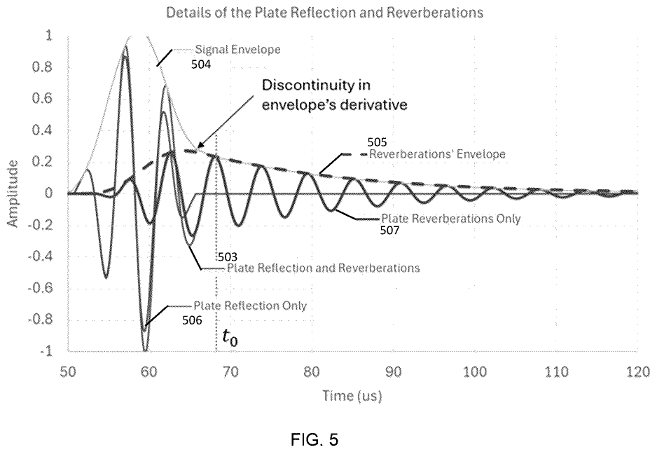

In FIG. 5 we show the signal recorded on transducer T1 (solid line 503) separated into plate reflection (solid line 506) and plate reverberations (solid line 507). As shown in this figure, at the envelope's derivative discontinuity the envelopes corresponding to the total signal (solid line 504) and reverberations only (dashed line 505) do not match.

The exact “matching” starts later and corresponds to one of the local peaks in the recoded signal amplitude. In practice calibration of the fluid cell device is required for the determination of the optimal to value.

Fluid Cell Device Design with Two Transducers

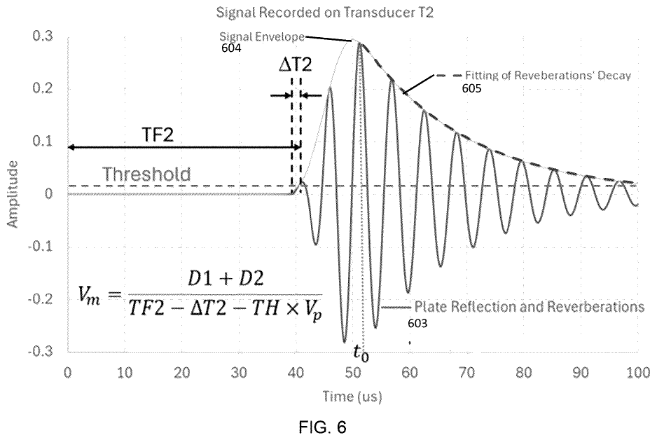

When two transducers are available, we get additional signal (acquired on the other side of the plate) to work with. FIG. 6 shows an example of such a signal (solid line 603) for the same wavelet and modeling parameters that were used in the previous examples and D2=0.5×D1. Also shown in this figure are the signal's envelope (solid line 604) and fitting of the decaying part of envelope (dashed line 605) with equation (8). As shown in FIG. 6, the mud velocity can be estimated according to the following:

$${V}_{m} = \frac{D 1 + D 2}{TF 2 - Δ T 2 - TH \times {V}_{p}} , \qquad ( 11 )$$

where Vₘ is the mud velocity, D1 and D2 are the widths of the two chambers of the fluid cell device, TF2 is the “time of flight” for the reflection signal, ΔT2 is the time offset accounting for noise threshold and time required for wavelet to gain the energy to raise above the noise level (one can get it from calibration), and TH×Vₚ is the time required for a signal to pass through plate. The estimation of mud impedance here is done similar to what was already described in the previous section, i.e., the reverberation decay is first fitted using equation (8) and then reflection coefficient R obtained in that fit is used for the calculation of mud impedance according to equation (10).

The use of the second transducer allows for a better resolution of the reverberations as its gain settings do not need to account for the arrival of reflected signal that is the largest in amplitude.

Additionally, according to the equations (5)-(7), the ratio between reverberations recorded by transducers T1 and T2 is equal to:

$${RV}_{n}^{T 1} / {RV}_{n}^{T 2} = R \times exp ( - α ( D 1 - D 2 ) ) , n = 1 , 2 , 3 , … \qquad ( 12 )$$

From the equation (12) mud attenuation a can be found using the following equation:

$$α = ln ( \frac{1}{R} \frac{{RV}_{n}^{T 1}}{{RV}_{n}^{T 2}} ) / ( {D}_{2} - {D}_{1} ) , n = 1 , 2 , 3 , … \qquad ( 13 )$$

We should note that equation (13) is based on our ability to determine where the “corresponding” reverberations in traces recorded on transducers T1 and T2 reside and our ability to extract their amplitudes. In general, it is not an easy task to implement. Instead, we propose using equation (8) to do that. We note, that if this relationship was used to determine coefficients for reverberations' decays on transducers T1 and T2 we need to shift one relative to the other by T₁₂ along the time axis to do “reverberations' alignment”. As such, we can use the following equations:

$${E}_{1} ( t ) = {a}_{1} \times {R}^{\frac{t}{TH * {V}_{p}}} , {E}_{2} ( t - {T}_{12} ) = {a}_{2} \times {R}^{\frac{- {T}_{12}}{TH * {V}_{p}}} \times {R}^{\frac{t}{TH * {V}_{p}}} , \qquad ( 14 )$$

where coefficients a₁ and a₂ were obtained using equation (8) using reverberations decays recorded by transducers T1 and T2. Substitution of relationships for E₁ and E₂ given by equation (14) instead of

$${RV}_{n}^{T 1} and {RV}_{n}^{T 2}$$

in equation (13) gives us an expression for mud attenuation which is based on coefficients a₁ and a₂:

$$α = ln ( {R}^{\frac{{T}_{12}}{TH * {V}_{p}} - 1} \times {a}_{1} / {a}_{2} ) / ( {D}_{2} - {D}_{1} ) . \qquad ( 15 )$$

Note that equation (15) assumes that transducers T1 and T2 have the same sensitivity. In practice, the transducers of the fluid cell device may need to be calibrated. Additionally, instead, or together with calibration, one may consider using transducer T2 as an acoustic source as well. In this case, we can use (15) to get 2 different mud attenuations estimations (when T1 is firing and T2 is firing). Averaging these 2 estimations provides a compensated measurement of mud attenuation that accounts for the difference in transducers' power and sensitivity.

### Additional Considerations

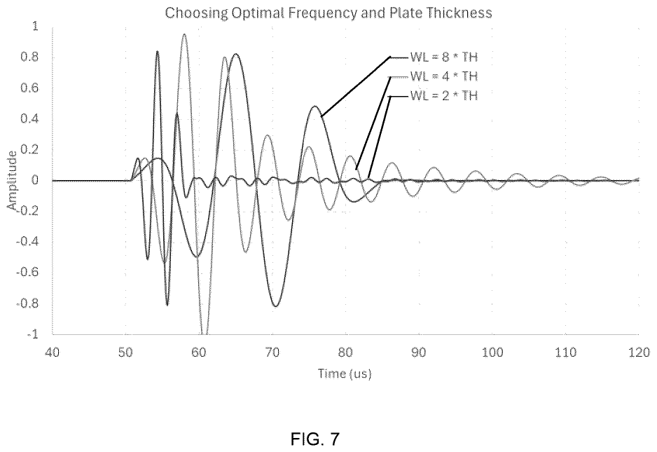

When building a fluid cell device based on the concepts described here one should choose the relationship between the transducer's central frequency and the plate thickness such that multiple reflections in the plate constructively interfere to provide a continuous reverberation tail for the mud impedance measurement. This can be achieved if the signal's wavelength in the plate is equal to four plate thicknesses, i.e. the following relationship holds true:

$$F = \frac{{V}_{p}}{4 \times TH} , \qquad ( 16 )$$

where F is the central frequency of the wavelet. As shown in FIG. 7, choosing the wavelength to be equal to four plate thicknesses (4*TH) produces a long continuous reverberation tail while other choices (8*TH and 2*TH)—result in destructive interference and mute reverberations.

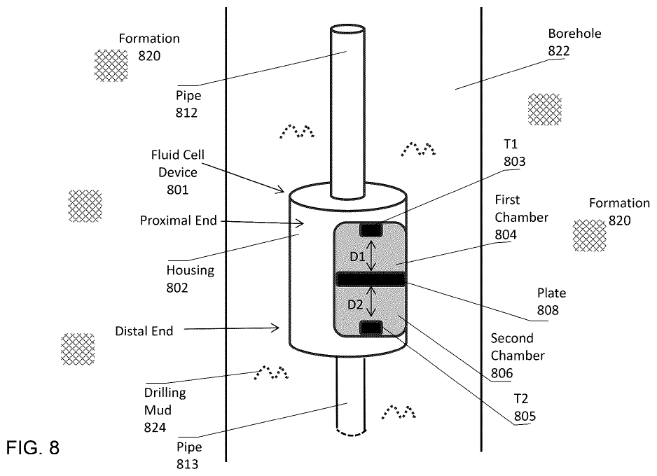

FIG. 8 illustrates an example of a fluid cell device in use in a borehole in accordance with the example embodiments described herein. The example of FIG. 8 illustrates a fluid cell device 801 that has been lowered into drilling mud 824 within a borehole 822 in a formation 820 of the earth. The fluid cell device 801 includes a housing 802, a top pipe 812 extending from a top of the housing 802, and a bottom pipe 813 extending from the bottom of the housing 802. The top and bottom pipes allow the fluid cell device 801 to be inserted between sections of pipe in a pipe string, such a drill pipe string. Alternatively, the fluid cell device 801 could be lowered into the drilling mud 824 on a wire line or other device. While not shown in FIG. 8, wiring can extend from housing 802 up through the top pipe 812 or a wireline to the surface of the borehole. Such wiring can be used to transmit data collected by the fluid cell device to a computing system at the surface for analysis.

Consistent with the previous embodiments described herein, the housing 802 includes a first chamber 804 and a second chamber 806 that are adjacent to each other and that are separated by a plate 808. In the example of FIG. 8, the first chamber 804 is located closer to the proximal end of the housing 802 and the second chamber 806 is located closer to the distal end of the housing 802. Although the first chamber 804 is located above the second chamber 806 in the example of FIG. 8, in other embodiments the first and second chambers can have other orientations such as a horizontal side by side configuration as long as the first and second chambers are adjacent and separated by a plate. Additionally, the housing 802 has a generally cylindrical shape in the example of FIG. 8. However, in alternate embodiments, the housing can have other shapes.

The first chamber 804 and the second chamber 806 are in the shape of recesses in the housing 802 into which the drilling mud 824 can flow. The first chamber 804 and the second chamber 806 may be completely separated by the plate 808 or the two chambers may be in fluid communication permitting drilling mud to flow between the two chambers. The first chamber 804 has a first transducer T1 803 located at a proximal end of the first chamber 804. At a distal end of the first chamber, the plate 808 is located with a proximal surface of the plate 808 facing the first transducer T1 803. Opposite the proximal surface of the plate 808 is the distal surface of the plate located at the proximal end of the second chamber 806 and facing a distal end of the second chamber. Optionally, at the distal end of the second chamber 806 a second transducer T2 805 can be located.

Consistent with the other embodiments herein, the fluid cell device 801 collects data relating to the drilling mud 824 in order to determine properties of the drilling mud 824. The first transducer T1 803 can emit an acoustic signal that will pass through drilling mud in the first chamber until it reaches the plate 808. The acoustic signal will generate reflection and reverberation signals when it impacts the plate 808. The reflection and reverberation signals can be detected by the first transducer T1 803. Alternatively or additionally, if the second transducer T2 805 is present, the second transducer T2 805 can be used to collect the reflection and reverberation signals.

The reflection and reverberation signals can be analyzed by a hardware processor located onboard the fluid cell device. Alternatively, the reflection and reverberation signals can be transmitted via a wired or wireless link to a hardware processor at the surface where they are analyzed. The reflection and reverberation signals can be analyzed to determine one or more properties of the drilling mud, including mud velocity, mud impedance, and mud attenuation. The determination of the drilling mud properties can be used to improve operations associated with the borehole. As one example, the determined drilling mud properties may be used to adjust the composition of the drilling mud. As another example, the determination of the drilling mud properties may be used to adjust drilling equipment in the borehole.

While particular embodiments are described above, it will be understood it is not intended to limit the invention to these particular embodiments. On the contrary, the invention includes alternatives, modifications and equivalents that are within the spirit and scope of the appended claims. Numerous specific details are set forth in order to provide a thorough understanding of the subject matter presented herein. But it will be apparent to one of ordinary skill in the art that the subject matter may be practiced without these specific details. In other instances, well-known methods, procedures, components, and circuits have not been described in detail so as not to unnecessarily obscure aspects of the embodiments.

The terminology used in the description of the invention herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, operations, elements, and/or components, but do not preclude the presence or addition of one or more other features, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

Although some of the various drawings illustrate a number of logical stages in a particular order, stages that are not order dependent may be reordered and other stages may be combined or broken out. While some reordering or other groupings are specifically mentioned, others will be obvious to those of ordinary skill in the art and so do not present an exhaustive list of alternatives. Moreover, it should be recognized that the stages could be implemented in hardware, firmware, software or any combination thereof.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A fluid cell device for measuring properties of a drilling mud in a borehole in an earth formation, the fluid cell device comprising:
a housing;
a first chamber disposed in the housing and having a first width, the first chamber configured to receive a first portion of the drilling mud;
a second chamber disposed in the housing adjacent to the first chamber and having a second width, the second chamber configured to receive a second portion of the drilling mud;
a plate disposed between the first chamber and the second chamber and at a distal end of the first chamber and a proximal end of the second chamber, a proximal surface of the plate facing the first chamber and a distal surface of the plate facing the second chamber; and
a first transducer disposed at a proximal end of the first chamber, wherein the first transducer directs an acoustic signal toward the proximal surface of the plate.

2. The fluid cell device of claim 1, wherein the first width extends from the first transducer to the distal end of the first chamber, and wherein the second width extends from distal surface of the plate to the distal end of the second chamber.

3. The fluid cell device of claim 2, wherein the first width and the second width are equal.

4. The fluid cell device of claim 2, wherein the first width and the second width are unequal.

5. The fluid cell device of claim 1, wherein the first transducer collects reflection and reverberation signals from the plate responsive to the acoustic signal.

6. The fluid cell device of claim 1, wherein a wavelength of the acoustic signal is four times a thickness of the plate.

7. The fluid cell device of claim 2, wherein a position of the plate is adjustable for varying the first width and the second width.

8. The fluid cell device of claim 1, further comprising a second transducer at a distal end of the second chamber, wherein the second transducer directs a second acoustic signal toward the distal surface of the plate.

9. The fluid cell device of claim 8, wherein the second transducer collects reflection and reverberation signals from the plate responsive to the second acoustic signal.

10. The fluid cell device of claim 1, wherein the housing comprises a first pipe extending from a top of the housing and a second pipe extending from the bottom of the housing.

11. A method for measuring properties of a drilling mud in a borehole in an earth formation, the method comprising:
deploying a fluid cell device into drilling mud in the borehole, the fluid cell device comprising:
a housing;
a first chamber disposed in the housing and having a first width, the first chamber configured to receive a first portion of the drilling mud;
a second chamber disposed in the housing adjacent to the first chamber and having a second width, the second chamber configured to receive a second portion of the drilling mud;
a plate disposed between the first chamber and the second chamber and at a distal end of the first chamber and a proximal end of the second chamber, a proximal surface of the plate facing the first chamber and a distal surface of the plate facing the second chamber; and
a first transducer disposed at a proximal end of the first chamber, wherein the first transducer directs an acoustic signal toward the proximal surface of the plate; and
analyzing recorded reflections and reverberations responsive to the acoustic signal to determine a mud velocity and a mud impedance of the drilling mud.

12. The method of claim 11, further comprising adjusting a composition of the drilling mud in response to determining the mud velocity and the mud impedance of the drilling mud.

13. The method of claim 11, further comprising adjusting a drilling tool in the borehole in response to determining the mud velocity and the mud impedance of the drilling mud.

14. The method of claim 11, wherein the fluid cell device further comprises a second transducer located at a distal end of the second chamber.

15. The method of claim 14, wherein the second transducer records second reflections and reverberations responsive to the acoustic signal.

16. The method of claim 14, wherein the second transducer records second reflections and reverberations response to a second acoustic signal emitted from the second transducer.

17. The method of claim 11, wherein a wavelength of the acoustic signal is four times a thickness of the plate.

18. The method of claim 11, wherein the housing is configured to be coupled to a drill string.

19. The method of claim 11, wherein the housing is configured to be coupled to a wire line.

20. The method of claim 11, further comprising analyzing the recorded reverberations responsive to the acoustic signal to determine a mud attenuation of the drilling mud.
